(19) United States
(12) Patent Application Publication
(10) Pub. No.: US 2025/0288382 A1
(43) Pub. Date: Sep. 18, 2025

(54) COMPUTER-IMPLEMENTED METHOD AND DEVICE CONFIGURED TO DETERMINE A DESIGN OF A MEDICAL DEVICE COMPRISING A ROD SHAPED PORTION

(71) Applicant: MAX-PLANCK-GESELLSCHAFT ZUR FÖRDERUNG DER WISSENSCHAFTEN E.V., München (DE)

(72) Inventors: Metin Sitti, Tübingen (DE); Martin Phelan, Tübingen (DE)

(73) Assignee: MAX-PLANCK-GESELLSCHAFT ZUR FÖRDERUNG DER WISSENSCHAFTEN E.V., München (DE)

(21) Appl. No.: 19/099,840

(22) PCT Filed: Aug. 1, 2023
(86) PCT No.: PCT/EP2023/071337

§ 371 Date: Jan. 30, 2025

(30) Foreign Application Priority Data
Aug. 2, 2022 (DE) 202022104403.1
Aug. 2, 2022 (DE) 202022104405.8
Aug. 2, 2022 (LU) LU502623
Aug. 2, 2022 (US) 17816774
Aug. 2, 2022 (US) 17816779
Dec. 9, 2022 (EP) 22212545.2
Dec. 9, 2022 (LU) LU503167

(51) Int. Cl. A61B 34/00; A61B 17/00; A61B 17/29; A61B 18/08; A61B 90/00; A61M 25/01

(52) U.S. Cl. CPC A61B 34/73; A61B 18/082; A61M 25/0127; A61B 2017/00296; A61B 2017/00411; A61B 2017/2932; A61B 2090/3762

(57) ABSTRACT

A method comprises simulating a shape of a rod-shaped portion resulting when at least one element located at a defined position at the rod-shaped portion having a defined size is subjected to a defined external magnetic field producing the external magnetic force using a FE model of the rod-shaped portion, determining a difference between the simulated shape and an at least one desired shape of the rod-shaped portion, the rod-shaped portion having the at least one desired shape being configured to be inserted into an anatomical structure of the human being and/or an animal, and adapting the defined position, the defined size and/or the defined external magnetic field based on the determined difference using an optimization method. The steps of simulating, determining, and adapting are carried out iteratively until the determined difference is below a defined threshold.

### CROSS REFERENCE TO RELATED APPLICATIONS

This application is a national stage of international patent application PCT/EP2023/071337, filed on Aug. 1, 2023 and designating the U.S., which claims priority to US patent application U.S. Ser. No. 17/816,774, filed on Aug. 2, 2022, and to German patent application DE 20 2022 104 403.1, filed on Aug. 2, 2022, and to US patent application U.S. Ser. No. 17/816,779, filed on Aug. 2, 2022, and to German patent application DE 20 2022 104 405.8, filed on Aug. 2, 2022, and to Luxembourgish patent application LU502623, filed on Aug. 2, 2022, and to European patent application EP 22 212 545.2, filed on Dec. 9, 2022, and to Luxembourgish patent application LU503167, filed on Dec. 9, 2022, all of which are hereby incorporated by reference in their entireties.

### TECHNICAL FIELD

The present disclosure is directed a computer-implemented method configured to determine a design of a medical device comprising a rod-shaped portion, a data processing device comprising a processor configured to carry out the method, a computer program comprising instructions which, when the program is executed by a computer, cause the computer to carry out the method, and a computer-readable medium comprising instructions which, when the instructions are executed by a computer, cause the computer to carry out the method.

### BACKGROUND

Any discussion of the related art throughout the specification should in no way be considered as an admission that such related art is widely known or forms part of common general knowledge in the field.

Wherever definitions of terms used in this description are given, the respective description is just one possible specific definition out of many possible definitions of the respective term and is thus not intended to limit the scope of the disclosure to this specific definition.

Cardiovascular disease (CVD) is the leading cause of death worldwide according to the World Health Organization, responsible for 17.9 million deaths as of 2019. Cardiovascular MRI (Magnetic Resonance Imaging) is a known modality for radiation-free diagnosis of cardiovascular complications such as congenital heart disease, heart failure, pericardial disease, and coronary heart disease. However, MR-guided interventional cardiology is a limited field of study due to the lack of MR-compatible tools that can safely operate within the MR environment and perform comparably to commercial catheters.

Conventionally an interventional cardiologist uses a commercial catheter shape with a distinct shape that is designed to assist in gaining access to a specific region of the heart of a human being and/or an animal during a catheterization procedure. However, it may be the case that the designated region cannot be accessed, e.g., due to the location of the affected area and/or the mechanical limitations of commercial catheters. This calls for either open surgery or recatheterization to introduce a superior shape for the application. However, open surgery increases patient recovery time and blood loss while recatheterization can add more time and radiation exposure to the intervention while keeping the patient under anesthesia.

Therefore, there is a growing need for MR-compatible catheters that can safely reach these regions of the heart without the need for additional tools.

### SUMMARY

A computer-implemented method configured for determining a design of a medical device comprising a rod-shaped portion configured to be inserted into an anatomical structure of a human being and/or an animal. The rod-shaped portion comprises at least one magnetic element configured and arranged to deform the rod-shaped portion using an external magnetic force acting on the at least one magnetic element. The method comprises a step of simulating a shape of the rod-shaped portion resulting when the at least one element located at a defined position at the rod-shaped portion having a defined size is subjected to a defined external magnetic field producing the external magnetic force using a finite element (FE) model of the rod-shaped portion. The method comprises a step of determining a difference between the simulated shape and at least one desired shape of the rod-shaped portion, the rod-shaped portion having at least one desired shape being configured to be inserted into the anatomical structure of the human being and/or the animal. The method comprises a step of adapting the defined position, the defined size and/or the defined external magnetic field based on the determined difference using an optimization method. The step of simulating, the step of determining and the step of adapting are carried out iteratively until the determined difference is below a defined threshold.

A medical device comprises a rod-shaped portion configured to be inserted into an anatomical structure of a human being and/or an animal. The rod-shaped portion comprises at least one magnetic element and/or at least one coil configured and arranged to deform the rod-shaped portion using an external magnetic force acting on the at least one magnetic element and/or the at least one coil. The rod-shaped portion comprises a design determined according to a method, the method including a step of simulating a shape of the rod-shaped portion resulting when the at least one element located at a defined position at the rod-shaped portion having a defined size is subjected to a defined external magnetic field producing the external magnetic force using a FE model of the rod-shaped portion; a step of determining a difference between the simulated shape and a at least one desired shape of the rod-shaped portion, the rod-shaped portion having the at least one desired shape being configured to be inserted into the anatomical structure of the human being and/or the animal; and a step of adapting the defined position, the defined size and/or the defined external magnetic field based on the determined difference using an optimization method; wherein the step of simulating, the step of determining and the step of adapting are carried out iteratively until the determined difference is below a defined threshold.

A computer-implemented method configured for determining a design of a medical device comprising a rod-shaped portion configured to be inserted into an anatomical structure of a human being and/or an animal, wherein the rod-shaped portion comprises at least one coil configured and arranged to deform the rod-shaped portion using a Lorentz force. The method comprises a step of simulating a shape of the rod-shaped portion resulting when the at least one coil located at a defined position at the rod-shaped portion having a defined size is supplied with a defined current while a defined external magnetic field acts on the coil using a FE model of the rod-shaped portion; a step of determining a difference between the simulated shape and at least one desired shape of the rod-shaped portion, the rod-shaped portion having the at least one desired shape being configured to be inserted into the anatomical structure of the human being and/or the animal; and a step of adapting the defined current, the defined position, the defined size and/or the defined external magnetic field based on the determined difference using an optimization method; wherein the step of simulating, the step of determining and the step of adapting are carried out iteratively until the determined difference is below a defined threshold.

Accordingly, the object is also solved by a computer-implemented method configured for determining a design of a medical device comprising a rod-shaped portion, optionally a catheter, such that the rod shaped portion is configured to be inserted into an anatomical structure, optionally a heart, of a human being and/or an animal, optionally through a blood vessel thereof.

The rod-shaped portion comprises at least one coil configured and arranged to deform the rod-shaped portion using a Lorentz force.

The method comprises a step of simulating a shape of the rod-shaped portion when the at least one coil located at a defined position at the rod-shaped portion having a defined size is supplied with a defined current while a defined external magnetic field acts on the coil using a FE model of the rod-shaped portion.

The method comprises a step of determining a difference between the simulated shape and a desired shape of the rod-shaped portion, the rod-shaped portion having the desired shape being configured to be inserted into the anatomical structure of the human being and/or the animal.

The method comprises a step of adapting the defined current, the defined position, the defined size and/or the defined external magnetic field based on the determined difference using an optimization method.

The step of simulating, the step of determining and the step of adapting are carried out iteratively until the determined difference is below a defined threshold.

Both methods are based on the same inventive idea, as will be discussed in detail below, wherein the first method is directed to a medical device (e.g., to be used in combination with an X-Ray imaging device) comprising a magnetic element permanently generating a static magnetic field and the second method is directed to a medical device (e.g., to be used in combination with an MRI device) comprising a coil configured to generate a magnetic field depending on a current supplied to the coil. However, both devices use magnetic forces to actuate or deform the rod-shaped portion. Therefore, if a description is given herein with respect to one of these specific designs, the description applies mutatis mutandis to the other design unless explicitly otherwise stated.

In other words, an automated design process for a technical system, i.e., the rod-shaped portion of the medical device, comprising at least one simulation step in which the behavior of the rod-shaped portion when being subjected to an external magnetic field is simulated, is provided.

As part of the simulation a finite element (FE) model is used to simulate the behavior, i.e., the deformation, of the rod-shaped portion caused by the Lorentz force. The desired shaped is chosen to be compatible with the intended use of the rod shape portion, i.e., to be suitable to be inserted into the anatomical structure, optionally into the blood vessel of a human being and/or an animal.

The underlying FE model forms in combination with the desired shape of the rod-shaped portion technical boundaries that contribute to technicality or the technical character of the claimed subject matter since they form the basis for a further technical use of the outcomes of the simulation, i.e., the resulting configuration/design of the rod-shaped portion of the medical being compatible with the anatomical structure in the magnetic field.

More specifically, the desired shape is limited by the physical reality, i.e., by the physical reality of the anatomical structure (e.g., by dimensions of blood vessels etc. in the anatomical structure), and the FE model is also limited by the physical reality, i.e., by the mechanical properties of the rod-shaped portion (e.g., length and stiffness thereof as well as the coil dimensions) and the magnetic field (e.g., the orientation of the magnetic field with respect to the at least one coil and the strength of the magnetic field). However, not only the models used in the method are based on technical principles, but they form the basis for a further technical use of the outcomes of the simulation, i.e., ensuring compatibility of the resulting design of the rod-shaped portion with the anatomical structure in combination with the magnetic field, and thus a use having an impact on the physical reality, wherein this use is explicitly specified in the independent claim. In conclusion, all the steps of the claimed method contribute to the technical character of the claimed subject matter.

Furthermore, the above described design process or method for determining the design the of the catheter based on the desired shape thereof goes below a straightforward automation of a method that may—at least in theory—be performed as a mental act but produces due to the specific implementation of the method a further technical effect.

More specifically, the method uses an optimization method to iteratively update/adapt/tune the input parameters (i.e., the defined current, the defined position, the defined size and/or the defined external magnetic field) thereby decreasing the number of iterations needed to find a design that is within the tolerance range, i.e., that achieves a difference below the threshold, in comparison to a straightforward implementation of randomly searching for a suitable design.

In the following optional further developments of the above described method are discussed in detail.

The method may comprise a step of determining a geometric model of an anatomical structure of a human being and/or an animal in which the rod-shaped portion should be inserted, and a step of determining the desired shape of the rod-shaped portion based on the determined geometric model.

The geometric model of the anatomical structure may be determined based on a CT (computer-assisted tomography/computed axial tomography) scan of the anatomical structure. The method may include a step of obtaining the CT scan using a CT scanner.

The method may comprise determining the FE model of the rod-shaped portion based on dimensions, e.g. a length and/or a diameter of the coils of the rod-shaped portion, and mechanical properties, e.g., a stiffness and/or elasticity of the rod-shaped portion, of the rod-shaped portion.

The method comprises outputting the defined current, the defined position, the defined size and/or the defined external magnetic field when the determined difference is below the predefined threshold.

That is, the defined current, the defined position, the defined size and/or the defined external magnetic field that were at last used by the method and that lead to a difference being below the predefined threshold may be output. This output data, i.e., the result of the method, may be considered functional data because it allows manufacturing the rod-shaped portion to be suitable for the intended use.

The method may comprise a step of manufacturing the rod-shaped portion of the medical device based on the output the defined size and/or the output defined position, a step of configuring a controller of the medical device configured to supply the at least one coil with a current based on the output defined current, and/or a step of configuring a controller of another medical device configured to generate a magnetic field based on the output defined external magnetic field.

The difference between the simulated shape and the desired shape of the rod-shaped portion is represented by a determined maximum deflection error among predefined measurement points on the desired shape and the simulated shape.

A deflection error may be defined as a distance between a predefined measurement point on the desired shape and a corresponding predefined measurement point on the simulated shape, wherein both measurements points have the same distance to a distal end of the rod-shaped portion in an initial state of the respective rod shape portion, i.e., in a state in which the rod shape portion is straight.

The optimization method may be based on a cost function mapping input parameters comprising the defined current, the defined position, the defined size and/or the defined external magnetic field to the determined maximum deflection error.

The optimization method may be based on an optimization problem searching for the arguments of the maxima of the cost function within a predefined search space for the defined current, the defined position, the defined size and/or the defined external magnetic field, respectively.

The step of simulating, the step of determining and the step of adapting may be carried out iteratively until the determined maximum deflection error is below the predefined threshold.

The method may comprise a step of determining the defined threshold based on dimensions of the rod-shaped portion, optionally based on a length of the rod-shaped portion, further optionally as a percentage of the length of the rod-shaped portion.

The method may comprise a step of determining the defined search space for the defined current, the defined position, the defined size and/or the defined external magnetic field based on physical limitations of the rod-shaped portion, respectively.

The defined size and/or the defined external magnetic field may be kept constant, respectively. Additionally or alternatively, a constant step size for adapting the defined current and/or the defined position may be used, respectively.

The rod shape portion of the medical device may be a catheter, optionally a cardiovascular catheter, configured to be inserted into a blood vessel of a human being and/or an animal, optional a distal end of the catheter. The catheter may be a coronary catheter.

The above description of the method may be summarized in other words with respect to a more concrete implementation of the disclosure as outlined in the following, wherein the following is not to be understood to limit the scope of the disclosure as defined by the claims, but is merely described for exemplary purposes.

Implementing multiple Lorentz Force-based coils on a catheter can enable increased dexterity for navigating confined workspaces, e.g., within the heart for treating cardio vascular diseases not limited to atrial fibrillation.

A simulation-based approach for designing such a multi-coil Lorentz forced-based cardiovascular catheter for intervening within MR scanners without the need for additional catheters may be provided.

Therefore, a finite element simulation developed to model the nonlinear deformations of the Lorentz forced-based catheter may be provided in combination with a design optimization problem that may be formulated to achieve a desired catheter shape using a Bayesian optimization algorithm.

In other words, this disclosure may take an approach in which Bayesian optimization using Gaussian Processes may be used for arbitrary catheter shapes, e.g., including known angiographic shapes. Therefore, the disclosure is not limited to generate a patient specific desired shape.

Bayesian optimization (BO) methods are advantageous due to their ability to test a small set of data points using a probabilistic model (Gaussian processes) to account for unknown variances in an experiment. However, the alternative or additional use of a genetic algorithm is also possible. The objective function may be tuned to the workspace of the heart in order to maximum coverage while maximizing contact force applied by the catheter on the heart surface to determine the optimal coil and catheter design.

Furthermore, a data processing device comprising a processor is provided, wherein the processor is configured to carry out the above described method at least partly.

Furthermore, a computer program is provided, wherein the computer program comprises instructions which, when the program is executed by a computer, cause the computer to carry out the above described method at least partly.

Furthermore, a computer-readable medium, optionally a computer-readable storage medium and/or a data signal (e.g., provided via the Internet), is provided, wherein the computer-readable medium comprises instructions which, when the instructions are executed by a computer, cause the computer to carry out the above described method at least partly.

Furthermore, a medical device comprising a rod-shaped portion configured to be inserted into an anatomical structure of a human being and/or an animal is provided. The rod-shaped portion comprises at least one magnetic element and/or at least one coil configured and arranged to deform the rod-shaped portion using an external magnetic force acting on the at least one magnetic element and/or the at least one coil. The rod-shaped portion comprises a design at least partly determined according to the above described method.

The above given description with respect to the method applies mutatis mutandis to the data processing device, the computer program, the computer-readable medium and/or, respectively, and vice versa.

The expression “computer-implemented method” covers claims which involve computers, computer networks or other programmable apparatus, whereby at least one feature is realized by means of a program. A computer-implemented method may be a method which is at least partly carried out by a data processing unit, e.g. a computer.

Instead of the term “current”, the term “electric current” may be used. Electric currents create magnetic fields, which may be used to actuate or move the medical device in the (external) magnetic field.

The term “determining” may include carrying out one or more mathematical operations in order to determine based on a given input in a given manner a desired output.

Instead of the term “coil”, the term “electromagnetic coil” may be used. An electromagnetic coil may be an electrical conductor such as a wire in the shape of a coil, spiral or helix. An electric current may be passed through the wire of the coil to generate a magnetic field. A current through any conductor creates a circular magnetic field around the conductor due to Ampere's law. One advantage of using the coil shape may be that it increases the strength of the magnetic field produced by a given current. The magnetic fields generated by the separate turns of wire all pass through the center of the coil and add (superpose) to produce a strong field there. The more turns of wire, the stronger the field produced may be and the stronger the effect of Joule heating may be.

The external magnetic field may be produced by an MRI device. The external magnetic field may have a field strength between 0.05 T and 25 T, optionally between 0.1 T and 21 T, further optionally between 0.1 T and 9,4 T, further optionally between 1,5 T and 7,0 T, further optionally between 0.1 T and 3,0 T. The external magnetic field may be static.

An optimization problem may be described as the problem of finding substantially the best solution from all feasible solutions, here to find a solution that is below the threshold.

The device, especially the catheter, may be a medical device. A medical device may be any device intended to be used for medical purposes. According to one possible definition a medical device may be an instrument, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in man or other animals, and/or intended to affect the structure or any function of the body of man or other animals, and which does not achieve its primary intended purposes through chemical action within or on the body of man or other animals and which is not dependent upon being metabolized for the achievement of its primary intended purposes. The term “medical device” may or may not include software functions. According to another possible definition the term “medical device” may mean any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings or animals for the purpose of diagnosis, prevention, monitoring, treatment or alleviation of disease, diagnosis, monitoring, treatment, alleviation of or compensation for an injury or handicap, investigation, replacement or modification of the anatomy or of a physiological process, and/or control of conception, and which does not achieve its principal intended action in or on the human and/or animal body by pharmacological, immunological or metabolic means, but which may be assisted in its function by such means.

A rod-shaped portion may be a portion or part of the medical device that may have a substantially circular cross section. The rod-shaped portion may have cylindrical form wherein a height or length of the rod-shaped portion exceeds the diameter of the rod-shaped portion. The tip of the rod-shaped portion may be outermost part of the rod-shaped portion in a forward direction of the medical device, i.e., a distal end thereof. The rod-shaped portion may comprise or may be realized using a tube. The distal end may be called insertion tip of the medical device.

A catheter may comprise a thin tube, i.e. the rod-shaped portion, made from medical grade materials serving a broad range of functions. Catheters are medical devices that can be inserted in the body to treat diseases and/or perform a surgical procedure. By modifying the material or adjusting the way catheters are manufactured, it is possible to tailor catheters for cardiovascular, urological, gastrointestinal, neurovascular, and ophthalmic applications. The process of inserting a catheter is “catheterization”. A catheter may comprise a thin, flexible tube (“soft” catheter) through catheters are available in varying levels of stiffness depending on the application. This may be taken into account by the (Cosserat) model. The catheter may be configured to be inserted into a body cavity, duct, or vessel, brain, skin or adipose tissue. Functionally, the catheter may allow drainage, administration of fluids or gases, access by surgical instruments, and/or perform a wide variety of other tasks depending on the type of catheter. The catheter may be a so called probe used in preclinical or clinical research for sampling of lipophilic and hydrophilic compounds, protein-bound and unbound drugs, neurotransmitters, peptides and proteins, antibodies, nanoparticles and nanocarriers, enzymes and vesicles.

A magnetic resonance imaging device is a medical imaging device configured to be used in magnetic resonance imaging (MRI) which is a medical imaging technique that may be used in radiology to form pictures of the anatomy and the physiological processes of the body. MRI scanners use strong magnetic fields, magnetic field gradients, and radio waves to generate images of the organs in the body.

More specifically, magnetic resonance imaging (MRI) is a medical imaging technique used in radiology to form pictures of the anatomy and/or the physiological processes of the body. MRI scanners use strong magnetic fields, magnetic field gradients, and radio waves to generate images, optionally of the organs in the body of a human being and/or an animal. MRI does not involve X-rays or the use of ionizing radiation, which distinguishes it from CT and PET scans. MRI is a medical application of nuclear magnetic resonance (NMR) which can also be used for imaging in other NMR applications, such as NMR spectroscopy.

The medical device, optionally the rod-shaped portion thereof, may be MRI compatible. Therefore, the medical device, optionally the rod-shaped portion thereof, may consist of non-magnetic material.

The term “consisting of” is exhaustive, i.e., the respective consisting of non-magnetic material does not comprise or contain magnetic material.

Non-magnetic material is any material that does not meet the definition of a magnetic material according to this disclosure. A magnetic material according to this disclosure is any material that may produce its own persistent magnetic field even in the absence of an applied magnetic field. Those materials are called magnets. Moreover, a magnetic material is any material that produces a magnetic field in response to an applied external magnetic field—a phenomenon known as magnetism—wherein the produced magnetic field is considerable in the context of or destroys MRI compatibility of the electric motor. More specifically, there are several types of magnetism, and all materials exhibit at least one of them. However, in this disclosure the term magnetism refers to ferromagnetic and ferrimagnetic materials only. These materials are the only ones that can retain magnetization and become magnets. Ferrimagnetic materials, which include ferrites and the magnetic materials magnetite and lodestone, are similar to but weaker than ferromagnetics. In other words, in this disclosure paramagnetic materials (such as platinum, aluminum, and oxygen) are considered non-magnetic material. Moreover, in this disclosure diamagnetic materials (such as carbon, copper, water, and plastic) which are repelled by both poles and which are compared to paramagnetic and ferromagnetic substances even more weakly repelled by a magnet are also considered non-magnetic materials.

Wherever the term “is configured to” in this disclosure is used, the term should be interpreted in a manner limiting the respective device which is configured to be used or to do something in that the device is structurally suitable or adapted to be used for the specific purpose. For example, in the present disclosure the medial device, especially the rod shape portion and more specifically the tip or distal end thereof, may be configured to be inserted into an anatomic structure, optionally a blood vessel, of an animal and/or a human being. Therefore, the rod-shaped portion may have dimensions that are limited by the dimensions of the anatomic structure, optionally an inner diameter of the blood vessel, of the human being and/or the animal in which it is configured to be inserted. To be configured to be inserted into the anatomic structure, optionally the blood vessel, the rod-shaped portion may have an outer diameter of 0.1 mm to 50 mm.

In one example, a multi-coil catheter, or MCC, may be constructed using a 6 F polyurethane tube and solenoid coils wrapped using 80 μm enameled copper wire with 150 coil loops. To pass wires through an inner channel of the MCC, all solenoids may be wound on separate tubing and reattached using heat shrink to form the catheter. Wire leads may be soldered using 400 μm copper wire.

Wherever the expressions “for example”, “by way of example”, “such as”, “e.g.” and the like are used, this shall be construed as being followed by the expression “and without limitation”, unless expressly stated otherwise. Similarly, “an example,” “exemplary,” and the like shall be construed as not limiting or as a non-exhaustive list.

For numerical indications, they are to be understood as both conclusive and non-exhaustive, i.e., for example, “a device” is to be understood as “at least one device and/or exactly one device”.

The term “substantially” allows for variations that do not adversely affect the intended purpose. Descriptive terms shall be understood to be modified by the term “substantially” even if the term “substantially” is not expressly stated.

The terms “comprising” and “including” and “with” and “having” (and similarly “comprises,” and “includes,” and “has”) and the like are used interchangeably and have the same meaning.

Consequently, unless the context clearly or explicitly requires otherwise, the words “comprising,” “including,” and the like in the description and claims are to be understood in an inclusive sense and not in an exclusive or exhaustive sense, i.e., in the sense of “including but not limited to.”

### BRIEF DESCRIPTION OF THE DRAWINGS

FIG. 1 schematically shows a flow-chart of a computer-implemented method according to the disclosure configured for determining a configuration of a medical device comprising a rod-shaped portion according to the disclosure, and

FIG. 2 schematically shows a perspective view on a catheter whose configuration/design is to be determined using said computer-implemented method.

### DESCRIPTION

One non-limiting example of a possible or an optional object of the disclosure can be formulated as to provide a computer-implemented method configured for determining an individual configuration/design of an MRI compatible catheter configured to reach a specific region of the heart of a human being and/or an animal during a catheterization procedure.

Accordingly, the object may be solved by a computer-implemented method configured for determining a design of a medical device comprising a rod-shaped portion, optionally a catheter, such that the rod-shaped portion is configured to be inserted into an anatomical structure, optionally a heart, of a human being and/or an animal, optionally through a blood vessel thereof.

The rod-shaped portion comprises at least one coil configured and arranged to deform the rod-shaped portion using a Lorentz force.

The method comprises a step of simulating a shape of the rod-shaped portion when the at least one coil located at a defined position at the rod-shaped portion having a defined size is supplied with a defined current while a defined external magnetic field acts on the coil using a FE model of the rod-shaped portion.

The method comprises a step of determining a difference between the simulated shape and a desired shape of the rod-shaped portion, the rod-shaped portion having the desired shape being configured to be inserted into the anatomical structure of the human being and/or the animal.

The method comprises a step of adapting the defined current, the defined position, the defined size and/or the defined external magnetic field based on the determined difference using an optimization method.

The step of simulating, the step of determining and the step of adapting are carried out iteratively until the determined difference is below a defined threshold.

Both methods are based on the same inventive idea, as will be discussed in detail below, wherein the first method is directed to a medical device (e.g., to be used in combination with an X-Ray imaging device) comprising a magnetic element permanently generating a static magnetic field and the second method is directed to a medical device (e.g., to be used in combination with an MRI device) comprising a coil configured to generate a magnetic field depending on a current supplied to the coil. However, both devices use magnetic forces to actuate or deform the rod-shaped portion. Therefore, if a description is given herein with respect to one of these specific designs, the description applies mutatis mutandis to the other design unless explicitly otherwise stated.

In other words, an automated design process for a technical system, i.e., the rod-shaped portion of the medical device, comprising at least one simulation step in which the behavior of the rod-shaped portion when being subjected to an external magnetic field is simulated, is provided.

As part of the simulation a finite element (FE) model is used to simulate the behavior, i.e., the deformation, of the rod-shaped portion caused by the Lorentz force. The desired shape is chosen to be compatible with the intended use of the rod shape portion, i.e., to be suitable to be inserted into the anatomical structure, optionally into the blood vessel of a human being and/or an animal.

The underlying FE model forms in combination with the desired shape of the rod-shaped portion technical boundaries that contribute to the technical character of the claimed subject matter since they form the basis for a further technical use of the outcomes of the simulation, i.e., the resulting configuration/design of the rod-shaped portion of the medical being compatible with the anatomical structure in the magnetic field.

More specifically, the desired shape is limited by the physical reality, i.e., by the physical reality of the anatomical structure (e.g., by dimensions of blood vessels etc. in the anatomical structure), and the FE model is also limited by the physical reality, i.e., by the mechanical properties of the rod-shaped portion (e.g., length and stiffness thereof as well as the coil dimensions) and the magnetic field (e.g., the orientation of the magnetic field with respect to the at least one coil and the strength of the magnetic field). However, not only the models used in the method are based on technical principles, but they form the basis for a further technical use of the outcomes of the simulation, i.e., ensuring compatibility of the resulting design of the rod-shaped portion with the anatomical structure in combination with the magnetic field, and thus a use having an impact on the physical reality, wherein this use is explicitly specified in the independent claim. In conclusion, all the steps of the claimed method contribute to the technical character of the claimed subject matter.

Furthermore, the above-described design process or method for determining the design the of the catheter based on the desired shape thereof goes below a straightforward automation of a method that may—at least in theory—be performed as a mental act but produces due to the specific implementation of the method a further technical effect.

More specifically, the method uses an optimization method to iteratively update/adapt/tune the input parameters (i.e., the defined current, the defined position, the defined size and/or the defined external magnetic field) thereby decreasing the number of iterations needed to find a design that is within the tolerance range, i.e., that achieves a difference below the threshold, in comparison to a straightforward implementation of randomly searching for a suitable design.

In the following, optional further developments of the above described method are discussed in detail.

The method may comprise a step of determining a geometric model of an anatomical structure of a human being and/or an animal in which the rod-shaped portion should be inserted, and a step of determining the desired shape of the rod-shaped portion based on the determined geometric model.

The geometric model of the anatomical structure may be determined based on a CT (computer-assisted tomography/computed axial tomography) scan of the anatomical structure. The method may include a step of obtaining the CT scan using a CT scanner.

The method may comprise determining the FE model of the rod-shaped portion based on dimensions, e.g., a length and/or a diameter of the coils of the rod-shaped portion, and mechanical properties, e.g., a stiffness and/or elasticity of the rod-shaped portion, of the rod-shaped portion.

The method comprises outputting the defined current, the defined position, the defined size and/or the defined external magnetic field when the determined difference is below the predefined threshold.

That is, the defined current, the defined position, the defined size and/or the defined external magnetic field that were at last used by the method and that lead to a difference being below the predefined threshold may be output. This output data, i.e., the result of the method, may be considered functional data because it allows manufacturing the rod-shaped portion to be suitable for the intended use.

The method may comprise a step of manufacturing the rod-shaped portion of the medical device based on the output the defined size and/or the output defined position, a step of configuring a controller of the medical device configured to supply the at least one coil with a current based on the output defined current, and/or a step of configuring a controller of another medical device configured to generate a magnetic field based on the output defined external magnetic field.

The difference between the simulated shape and the desired shape of the rod-shaped portion is represented by a determined maximum deflection error among predefined measurement points on the desired shape and the simulated shape.

A deflection error may be defined as a distance between a predefined measurement point on the desired shape and a corresponding predefined measurement point on the simulated shape, wherein both measurements points have the same distance to a distal end of the rod-shaped portion in an initial state of the respective rod shape portion, i.e., in a state in which the rod shape portion is straight.

The optimization method may be based on a cost function mapping input parameters comprising the defined current, the defined position, the defined size and/or the defined external magnetic field to the maximum deflection error.

The optimization method may be based on an optimization problem searching for the arguments of the maxima of the cost function within a predefined search space for the defined current, the defined position, the defined size and/or the defined external magnetic field, respectively.

The step of simulating, the step of determining and the step of adapting may be carried out iteratively until the determined maximum deflection error is below the predefined threshold.

The method may comprise a step of determining the defined threshold based on dimensions of the rod-shaped portion, optionally based on a length of the rod-shaped portion, further optionally as a percentage of the length of the rod-shaped portion.

The method may comprise a step of determining the defined search space for the defined current, the defined position, the defined size and/or the defined external magnetic field based on physical limitations of the rod-shaped portion, respectively.

The defined size and/or the defined external magnetic field may be kept constant, respectively. Additionally or alternatively, a constant step size for adapting the defined current and/or the defined position may be used, respectively.

The rod shape portion of the medical device may be a catheter, optionally a cardiovascular catheter, configured to be inserted into a blood vessel of a human being and/or an animal, optional a distal end of the catheter. The catheter may be a coronary catheter.

The above description of the method may be summarized in other words with respect to a more concrete implementation of the disclosure as outlined in the following, wherein the following is not to be understood to limit the scope of the disclosure as defined by the claims, but is merely described for exemplary purposes.

Implementing multiple Lorentz Force-based coils on a catheter can enable increased dexterity for navigating confined workspaces, e.g., within the heart for treating cardio vascular diseases not limited to atrial fibrillation.

A simulation-based approach for designing such a multi-coil Lorentz forced-based cardiovascular catheter for intervening within MR scanners without the need for additional catheters may be provided.

Therefore, a finite element simulation developed to model the nonlinear deformations of the Lorentz forced-based catheter may be provided in combination with a design optimization problem that may be formulated to achieve a desired catheter shape using a Bayesian optimization algorithm.

In other words, this disclosure may take an approach in which Bayesian optimization using Gaussian Processes may be used for arbitrary catheter shapes, e.g., including known angiographic shapes. Therefore, the disclosure is not limited to generating a patient specific desired shape.

Bayesian optimization (BO) methods are advantageous due to their ability to test a small set of data points using a probabilistic model (Gaussian processes) to account for unknown variances in an experiment. However, the alternative or additional use of a genetic algorithm is also possible. The objective function may be tuned to the workspace of the heart in order to maximum coverage while maximizing contact force applied by the catheter on the heart surface to determine the optimal coil and catheter design.

Furthermore, a data processing device comprising a processor is provided, wherein the processor is configured to carry out the above-described method at least partly.

Furthermore, a computer program is provided, wherein the computer program comprises instructions which, when the program is executed by a computer, cause the computer to carry out the above-described method at least partly.

Furthermore, a computer-readable medium, optionally a computer-readable storage medium and/or a data signal (e.g., provided via the Internet), is provided, wherein the computer-readable medium comprises instructions which, when the instructions are executed by a computer, cause the computer to carry out the above-described method at least partly.

Furthermore, a medical device comprising a rod-shaped portion configured to be inserted into an anatomical structure of a human being and/or an animal is provided. The rod-shaped portion comprises at least one magnetic element and/or at least one coil configured and arranged to deform the rod-shaped portion using an external magnetic force acting on the at least one magnetic element and/or the at least one coil. The rod-shaped portion comprises a design at least partly determined according to the above-described method.

The above given description with respect to the method applies mutatis mutandis to the data processing device, the computer program, the computer-readable medium and/or, respectively, and vice versa.

The expression “computer-implemented method” covers claims which involve computers, computer networks or other programmable apparatus, whereby at least one feature is realized by means of a program. A computer-implemented method may be a method which is at least partly carried out by a data processing unit, e.g. a computer.

Instead of the term “current”, the term “electric current” may be used. Electric currents create magnetic fields, which may be used to actuate or move the medical device in the (external) magnetic field.

The term “determining” may include carrying out one or more mathematical operations in order to determine based on a given input in a given manner a desired output.

Instead of the term “coil”, the term “electromagnetic coil” may be used. An electromagnetic coil may be an electrical conductor such as a wire in the shape of a coil, spiral or helix. An electric current may be passed through the wire of the coil to generate a magnetic field. A current through any conductor creates a circular magnetic field around the conductor due to Ampere's law. One advantage of using the coil shape may be that it increases the strength of the magnetic field produced by a given current. The magnetic fields generated by the separate turns of wire all pass through the center of the coil and add (superpose) to produce a strong field there. The more turns of wire, the stronger the field produced may be and the stronger the effect of Joule heating may be.

The external magnetic field may be produced by an MRI device. The external magnetic field may have a field strength between 0.05 T and 25 T, optionally between 0.1 T and 21 T, further optionally between 0.1 T and 9.4 T, further optionally between 1.5 T and 7.0 T, further optionally between 0.1 T and 3.0 T. The external magnetic field may be static.

An optimization problem may be described as the problem of finding substantially the best solution from all feasible solutions, here to find a solution that is below the threshold.

The device, especially the catheter, may be a medical device. A medical device may be any device intended to be used for medical purposes. According to one possible definition a medical device may be an instrument, apparatus, implement, machine, contrivance, implant, in vitro reagent, or other similar or related article, including a component part, or accessory which is intended for use in the diagnosis of disease or other conditions, or in the cure, mitigation, treatment, or prevention of disease, in man or other animals, and/or intended to effect the structure or any function of the body of man or other animals, and which does not achieve its primary intended purposes through chemical action within or on the body of man or other animals and which is not dependent upon being metabolized for the achievement of its primary intended purposes. The term “medical device” may or may not include software functions. According to another possible definition the term “medical device” may mean any instrument, apparatus, appliance, software, material or other article, whether used alone or in combination, including the software intended by its manufacturer to be used specifically for diagnostic and/or therapeutic purposes and necessary for its proper application, intended by the manufacturer to be used for human beings or animals for the purpose of diagnosis, prevention, monitoring, treatment or alleviation of disease, diagnosis, monitoring, treatment, alleviation of or compensation for an injury or handicap, investigation, replacement or modification of the anatomy or of a physiological process, and/or control of conception, and which does not achieve its principal intended action in or on the human and/or animal body by pharmacological, immunological or metabolic means, but which may be assisted in its function by such means.

A rod-shaped portion may be a portion or part of the medical device that may have a substantially circular cross section. The rod-shaped portion may have cylindrical form wherein a height or length of the rod-shaped portion exceeds the diameter of the rod-shaped portion. The tip of the rod-shaped portion may be outermost part of the rod-shaped portion in a forward direction of the medical device, i.e., a distal end thereof. The rod-shaped portion may comprise or may be realized using a tube. The distal end may be called an insertion tip of the medical device.

A catheter may comprise a thin tube, i.e. the rod-shaped portion, made from medical grade materials serving a broad range of functions. Catheters are medical devices that can be inserted in the body to treat diseases and/or perform a surgical procedure. By modifying the material or adjusting the way catheters are manufactured, it is possible to tailor catheters for cardiovascular, urological, gastrointestinal, neurovascular, and ophthalmic applications. The process of inserting a catheter is “catheterization”. A catheter may comprise a thin, flexible tube (“soft” catheter) though catheters are available in varying levels of stiffness depending on the application. This may be taken into account by the (Cosserat) model. The catheter may be configured to be inserted into a body cavity, duct, or vessel, brain, skin or adipose tissue. Functionally, the catheter may allow drainage, administration of fluids or gases, access by surgical instruments, and/or perform a wide variety of other tasks depending on the type of catheter. The catheter may be a so called probe used in preclinical or clinical research for sampling of lipophilic and hydrophilic compounds, protein-bound and unbound drugs, neurotransmitters, peptides and proteins, antibodies, nanoparticles and nanocarriers, enzymes and vesicles.

A magnetic resonance imaging device is a medical imaging device configured to be used in magnetic resonance imaging (MRI) which is a medical imaging technique that may be used in radiology to form pictures of the anatomy and the physiological processes of the body. MRI scanners use strong magnetic fields, magnetic field gradients, and radio waves to generate images of the organs in the body.

More specifically, magnetic resonance imaging (MRI) is a medical imaging technique used in radiology to form pictures of the anatomy and/or the physiological processes of the body. MRI scanners use strong magnetic fields, magnetic field gradients, and radio waves to generate images, optionally of the organs in the body of a human being and/or an animal. MRI does not involve X-rays or the use of ionizing radiation, which distinguishes it from CT and PET scans. MRI is a medical application of nuclear magnetic resonance (NMR) which can also be used for imaging in other NMR applications, such as NMR spectroscopy.

The medical device, optionally the rod-shaped portion thereof, may be MRI compatible. Therefore, the medical device, optionally the rod-shaped portion thereof, may consist of non-magnetic material.

The term “consisting of” is exhaustive, i.e., the respective consisting of non-magnetic material does not comprise or contain magnetic material.

Non-magnetic material is any material that does not meet the definition of a magnetic material according to this disclosure. A magnetic material according to this disclosure is any material that may produce its own persistent magnetic field even in the absence of an applied magnetic field. Those materials are called magnets. Moreover, a magnetic material is any material that produces a magnetic field in response to an applied external magnetic field—a phenomenon known as magnetism—wherein the produced magnetic field is considerable in the context of or destroys MRI compatibility of the electric motor. More specifically, there are several types of magnetism, and all materials exhibit at least one of them. However, in this disclosure the term magnetism refers to ferromagnetic and ferrimagnetic materials only. These materials are the only ones that can retain magnetization and become magnets. Ferrimagnetic materials, which include ferrites and the magnetic materials magnetite and lodestone, are similar to but weaker than ferromagnetic materials. In other words, in this disclosure paramagnetic materials (such as platinum, aluminum, and oxygen) are considered non-magnetic materials. Moreover, in this disclosure diamagnetic materials (such as carbon, copper, water, and plastic) which are repelled by both poles and which are compared to paramagnetic and ferromagnetic substances even more weakly repelled by a magnet are also considered non-magnetic materials.

Wherever the term “is configured to” in this disclosure is used, the term should be interpreted in a manner limiting the respective device which is configured to be used or to do something in that the device is structurally suitable or adapted to be used for the specific purpose. For example, in the present disclosure the medial device, especially the rod shape portion and more specifically the tip or distal end thereof, may be configured to be inserted into an anatomic structure, optionally a blood vessel, of an animal and/or a human being. Therefore, the rod-shaped portion may have dimensions that are limited by the dimensions of the anatomic structure, optionally an inner diameter of the blood vessel, of the human being and/or the animal in which it is configured to be inserted. To be configured to be inserted into the anatomic structure, optionally the blood vessel, the rod-shaped portion may have an outer diameter of 0.1 mm to 50 mm.

In one example, a multi-coil catheter, or MCC, may be constructed using a 6 F polyurethane tube and solenoid coils wrapped using 80 μm enameled copper wire with 150 coil loops. To pass wires through an inner channel of the MCC, all solenoids may be wound on separate tubing and reattached using heat shrink to form the catheter. Wire leads may be soldered using 400 μm copper wire.

Wherever the expressions “for example”, “by way of example”, “such as”, “e.g.” and the like are used, this shall be construed as being followed by the expression “and without limitation”, unless expressly stated otherwise. Similarly, “an example,” “exemplary,” and the like shall be construed as not limiting or as a non-exhaustive list.

For numerical indications, they are to be understood as both conclusive and non-exhaustive, i.e., for example, “a device” is to be understood as “at least one device and/or exactly one device”.

The term “substantially” allows for variations that do not adversely affect the intended purpose. Descriptive terms shall be understood to be modified by the term “substantially” even if the term “substantially” is not expressly stated.

The terms “comprising” and “including” and “with” and “having” (and similarly “comprises,” and “includes,” and “has”) and the like are used interchangeably and have the same meaning.

Consequently, unless the context clearly or explicitly requires otherwise, the words “comprising,” “including,” and the like in the description and claims are to be understood in an inclusive sense and not in an exclusive or exhaustive sense, i.e., in the sense of “including but not limited to.”

As required, a detailed embodiment is disclosed herein; however, it is to be understood that the disclosed embodiment is merely exemplary of the disclosure that may be embodied in various forms. The figures are not necessarily to scale, and some features may be exaggerated to show details of particular components. Therefore, specific structural and functional details disclosed in the figures are not to be interpreted as limiting, but merely as a representative basis for teaching one skilled in the art to variously employ the present disclosure.

Throughout the figures the same components are indicated by the same reference signs.

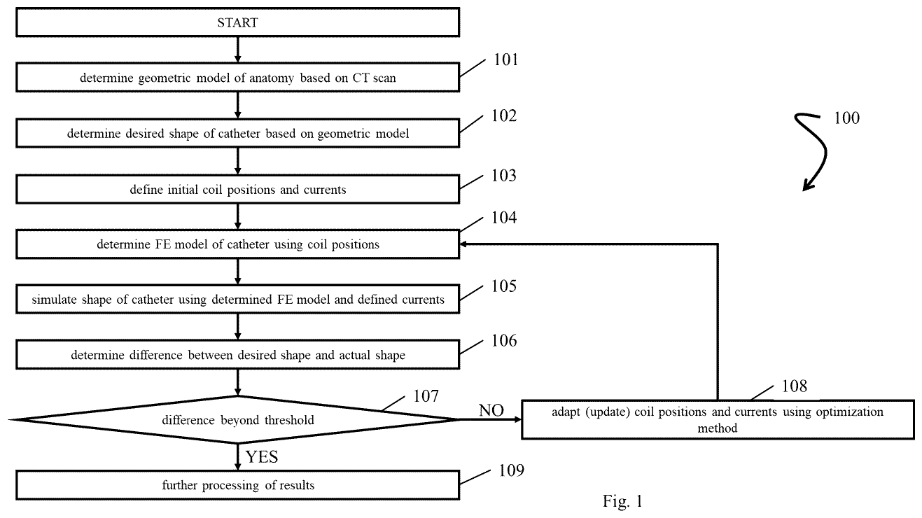

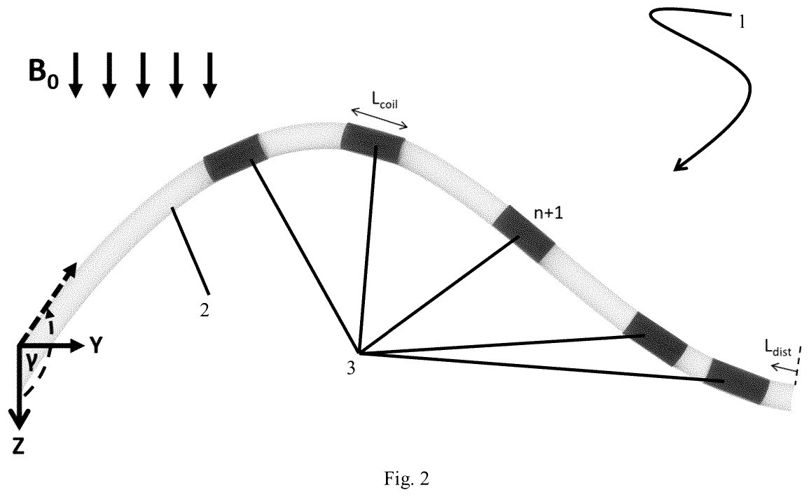

In FIG. 1 a flowchart for a computer-implemented method 100 configured for determining a design of a medical device 1 comprising a rod-shaped portion 2 (see FIG. 2) being configured to be inserted into an anatomical structure of a human being and/or an animal is shown.

As can be gathered from FIG. 1, the flexible/elastic rod-shaped portion 2 comprises at least one coil (or magnetic element) 3 configured and arranged to deform the rod-shaped portion using an external magnetic field B₀ acting on the at least one coil (magnetic element) 3 resulting in case the coil 3 is used a Lorentz force. In the specific example shown in FIG. 2, five coils (or magnetic elements) 3 are used. In the following the embodiment focuses on the rod-shaped portion 2 being equipped with coils 3, but (as already explained above) the following description applies mutatis mutandis.

The method comprises an optional step 101 of determining a geometric model of the anatomical structure of the human being and/or the animal.

The method comprises an optional step 102 of determining the desired shape of the rod-shaped portion 2 based on the determined geometric model.

The geometric model of the anatomical structure may be determined based on a CT scan of the anatomical structure.

Steps 101 and 102 allow for a patient specific determination of the desired shape. However, additionally or alternatively, one or more desired shapes for the rod-shaped portion may be loaded from a database comprising the one or more desired shapes, e.g., commercial catheter shapes widely used in cardiovascular catheterization.

The method comprises an optional step 103 of defining an initial coil position, an initial current and/or coil size for each one of the coils 3, respectively, and an initial magnetic field strength and/or orientation of the external magnetic field B₀.

The method comprises an optional step 104 of (initially) determining a FE model of the rod-shaped portion 2 based on dimensions and mechanical properties of the rod-shaped portion.

The method comprises a step 105 of simulating a shape of the rod-shaped portion 2 resulting when the coils 3 located at the respective defined position at the rod-shaped portion 2 having the respective defined size are supplied with the respective defined current while the respective defined external magnetic field B₀ acts on the coils 3 using the FE model of the rod-shaped portion 2.

More specifically, the finite element (FE) model may solve the deformation of the rod-shaped portion, e.g., a (soft) catheter, driven by the Lorentz force, which is generated by the current-carrying coils 3 in a (high) magnetic field such within an MRI device. The model may be implemented using a FE solver and a code written to simulate magnetic torques applied on the rod-shaped portion 2. It may be assumed that the rod-shaped portion 2 is oriented at 90° with respect to a static magnetic field B₀ to maximize the output torque from the coils 3 and to simplify the following search for suitable parameters. The same is true for the coil size and/or the number of coils, which is fixed to five coils 3 in this specific example. In this case, solely the coil positions and the respective current supplied to the coils 3 is optimized. The same is also true for the desired shape, wherein it is also possible to use multiple shapes and determine one design of the rod-shaped portion 2 with respect to coil size, coil number, coil position and/or magnetic field that fits all these desired shapes when varying the current supplied to the respective coils.

The method comprises a step 106 of determining a difference between the simulated shape and a desired shape of the rod-shaped portion 2, the rod-shaped portion 2 having the desired shape being configured to be inserted into the anatomical structure of the human being and/or the animal.

The method comprises a step 105 of determining a difference between the simulated shape and a desired shape of the rod-shaped portion 2, the rod-shaped portion 2 having the desired shape being configured to be inserted into the anatomical structure of the human being and/or the animal.

The method comprises a step 107 of determining if a difference between the simulated shape and a desired shape of the rod-shaped portion 2 is below a defined threshold.

The difference between the simulated shape and the desired shape of the rod-shaped portion 2 may be represented by a maximum deflection error among predefined measurement points on the desired shape and the simulated shape.

If the difference is below the threshold, the method comprises a step 108 of adapting the defined current, the defined position, the defined size and/or the defined external magnetic field B₀ based on the determined difference using an optimization method. A constant step size for adapting the defined current, the defined size, the defined external magnetic field B₀ and/or the defined position may be used, respectively.

The optimization method may be based on a cost function mapping input parameters comprising the defined current, the defined position, the defined size and/or the defined external magnetic field to the maximum deflection error.

The optimization method may be based on an optimization problem searching for the arguments of the maxima of the cost function within a predefined search space for the defined current, the defined position, the defined size and/or the defined external magnetic field, respectively.

The step 108 may comprise an optional step of determining the defined search space for the defined current, the defined position, the defined size and/or the defined external magnetic field based on physical limitations of the rod-shaped portion 2, respectively.

More specifically, since this disclosure aims to find—inter alia—the design and actuation parameters of, e.g., an MRI-driven catheter to generate a desired/given shape, a cost function of the proposed optimization method may be defined as:

D:Θ→

[symbol]

which maps the input parameters to a scalar cost value, (i.e. the maximum deflection error among the measurement points on the desired and simulated shapes). The input parameter θ=[d, I] may consist of the position of n many coils on the catheter, dϵRⁿ, and the current values, IϵRⁿ, running on them. On the other hand, the number of coils and their sizes may be kept constant and set to for example 3 and 5 mm (e.g., corresponding to 150 turns), respectively, to limit the search space size.

Based on this cost function, the optimization problem may be formulated as:

$${θ}^{*} = {arg max D ( θ )}_{θϵΘ}$$

where Θ and D(θ) are the complete search space and the obtained deflection error for a given input parameter set θ, respectively. The proposed optimization method may iteratively test input parameters until the maximum error reaches the target value of, for example, 2% of the catheter's length (e.g., defined as 15 cm). While defining the search space, the ranges of the input parameters may be determined based on the physical limitations of the catheter design. Accordingly, the coil currents may be defined between-300 mA and 300 mA, whereas the limits of each coil's position may be found considering the total length of the catheter, coils and other coils' positions. The step sizes for coil currents and positions used to discretize the search space may be 10 mA and 5 mm (L_{coil}). Coil positions may be defined as the distance from the: 1) tip point of the catheter to the first coil (L_{dist}) and 2) end point of the (n+1th) coil to the (n^{th}) coil.

The results of the design algorithm indicate it is feasible to achieve a library of 100 shapes using various design inputs including coil locations and current inputs, wherein a study shows that the shapes may be achieved with less than 2% error and solved in less than 100 iterations (median: 10, average: 17.7, max: 95).

Then the method starts again with step 104 and updates or adapts the FE model based on the updated defined current, the defined position, the defined size and/or the defined external magnetic field B₀.

Therefore, the above described steps 104-108 are carried out iteratively until the determined difference is below a defined threshold, e.g., 2% of the length (e.g., of a defined part) of the rod-shaped portion 2. If the difference, e.g., the determined maximum deflection error, is below the defined or fixed threshold, the method moves forward with an optional step 109 of further processing the results of the method, i.e., the defined current, the defined position, the defined size and/or the defined external magnetic field B₀.

The optional step 109 may comprise a step of outputting the defined current, the defined position, the defined size and/or the defined external magnetic field, a step of manufacturing the rod-shaped portion of the medical device based on the output the defined size and/or the output defined position, a step of configuring a controller of the medical device configured to supply the at least one coil with a current based on the output defined current, and/or a step of configuring a controller of another medical device configured to generate a magnetic field based on the output defined external magnetic field B₀.

What is claimed is:

1-17. (canceled)

18. A computer-implemented method configured for determining a design of a medical device comprising a rod-shaped portion configured to be inserted into an anatomical structure of a human being and/or an animal, wherein the rod-shaped portion includes at least one magnetic element configured and arranged to deform the rod-shaped portion using an external magnetic force acting on the at least one magnetic element, the computer-implemented method comprising:
simulating a shape of the rod-shaped portion resulting when the at least one magnetic element located at a defined position at the rod-shaped portion having a defined size is subjected to a defined external magnetic field producing the external magnetic force using a FE model of the rod-shaped portion,
determining a difference between the simulated shape and an at least one desired shape of the rod-shaped portion, the rod-shaped portion having the at least one desired shape being configured to be inserted into the anatomical structure of the human being and/or the animal, and
adapting the defined position, the defined size and/or the defined external magnetic field based on the determined difference using an optimization method,
wherein the simulating, the determining, and the adapting are carried out iteratively until the determined difference is below a defined threshold.

19. The computer-implemented method according to claim 18, further comprising:
determining a geometric model of the anatomical structure of the human being and/or the animal, and
determining the at least one desired shape of the rod-shaped portion based on the determined geometric model.

20. The computer-implemented method according to claim 19, wherein the geometric model of the anatomical structure is determined based on a CT scan of the anatomical structure.

21. The computer-implemented method according to claim 18, further comprising determining the FE model of the rod-shaped portion based on dimensions and mechanical properties of the rod-shaped portion.

22. The computer-implemented method according to claim 18, further comprising outputting the defined position, the defined size and/or the defined external magnetic field when the determined difference is below a predefined threshold.

23. The computer-implemented method according to claim 22, further comprising manufacturing the rod-shaped portion of the medical device based on the output defined size and/or the output defined position.

24. The computer-implemented method according to claim 22, further comprising configuring a controller of another medical device configured to generate a magnetic field based on the output defined external magnetic field.

25. The computer-implemented method according to claim 18, wherein:
the difference between the simulated shape and the at least one desired shape of the rod-shaped portion is represented by a determined maximum deflection error among predefined measurement points on the at least one desired shape and the simulated shape,
the optimization method is based on a cost function mapping input parameters comprising the defined position, the defined size and/or the defined external magnetic field to the determined maximum deflection error, and
the optimization method is based on an optimization problem searching for arguments of a maxima of the cost function within a predefined search space for the defined position, the defined size and/or the defined external magnetic field, respectively.

26. The computer-implemented method according to claim 25, wherein the simulating, the determining and the adapting are carried out iteratively until the determined maximum deflection error is below a predefined threshold.

27. The computer-implemented method according to claim 26, wherein the method comprises determining the defined threshold based on dimensions of the rod-shaped portion, optionally based on a length of the rod-shaped portion, further optionally as a percentage of the length of the rod-shaped portion.

28. The computer-implemented method according to claim 25, further comprising determining the defined search space for the defined position, the defined size and/or the defined external magnetic field based on physical limitations of the rod-shaped portion, respectively.

29. The computer-implemented method according to claim 18, wherein:
the defined size and/or the defined external magnetic field are kept constant, respectively, and/or
a constant step size for adapting the defined position is used, respectively.

30. The computer-implemented method according to claim 18, wherein the rod-shaped portion of the medical device is a catheter configured to be inserted into a blood vessel of the human being and/or the animal.

31. A non-transitory computer-readable medium, wherein the computer-readable medium comprises instructions which, when the instructions are executed by a computer, cause the computer to carry out the method according to claim 18.

32. A medical device comprising a rod-shaped portion configured to be inserted into an anatomical structure of a human being and/or an animal, wherein:
the rod-shaped portion includes at least one magnetic element and/or at least one coil configured and arranged to deform the rod-shaped portion using an external magnetic force acting on the at least one magnetic element and/or the at least one coil, and
the rod-shaped portion comprises a design determined according to a method, the method including:
simulating a shape of the rod-shaped portion resulting when the at least one element located at a defined position at the rod-shaped portion having a defined size is subjected to a defined external magnetic field producing the external magnetic force using a FE model of the rod-shaped portion,
determining a difference between the simulated shape and an at least one desired shape of the rod-shaped portion, the rod-shaped portion having the at least one desired shape being configured to be inserted into the anatomical structure of the human being and/or the animal, and
adapting the defined position, the defined size and/or the defined external magnetic field based on the determined difference using an optimization method,
wherein the simulating, the determining and the adapting are carried out iteratively until the determined difference is below a defined threshold.

33. A computer-implemented method configured for determining a design of a medical device including a rod-shaped portion configured to be inserted into an anatomical structure of a human being and/or an animal, wherein the rod-shaped portion comprises at least one coil configured and arranged to deform the rod-shaped portion using a Lorentz force, the computer-implemented method comprising:
simulating a shape of the rod-shaped portion resulting when the at least one coil located at a defined position at the rod-shaped portion having a defined size is supplied with a defined current while a defined external magnetic field acts on the coil using a FE model of the rod-shaped portion,
determining a difference between the simulated shape and at least one desired shape of the rod-shaped portion, the rod-shaped portion having the at least one desired shape being configured to be inserted into the anatomical structure of the human being and/or the animal, and
adapting the defined current, the defined position, the defined size and/or the defined external magnetic field based on the determined difference using an optimization method,
wherein the simulating, the determining and the adapting are carried out iteratively until the determined difference is below a defined threshold.

34. The computer-implemented method according to claim 33, further comprising outputting the defined current, the defined position, the defined size and/or the defined external magnetic field when the determined difference is below a predefined threshold.

35. The computer-implemented method according to claim 34, further comprising configuring a controller of the medical device configured to supply the at least one coil with a current based on the output defined current.

36. The computer-implemented method according to claim 33, wherein:
the difference between the simulated shape and the at least one desired shape of the rod-shaped portion is represented by a determined maximum deflection error among predefined measurement points on the at least one desired shape and the simulated shape,
the optimization method is based on a cost function mapping input parameters comprising the defined current, the defined position, the defined size and/or the defined external magnetic field to the determined maximum deflection error, and
the optimization method is based on an optimization problem searching for arguments of a maxima of the cost function within a predefined search space for the defined current, the defined position, the defined size and/or the defined external magnetic field, respectively,
the method optionally further comprises determining the defined search space for the for the defined current, the defined position, the defined size and/or the defined external magnetic field based on physical limitations of the rod-shaped portion, respectively.

37. The computer-implemented method according to claim 33, wherein:
the defined size and/or the defined external magnetic field are kept constant, respectively, and/or
a constant step size for adapting the defined current and/or the defined position is used, respectively.
